(19) United States
(12) Patent Application Publication
(10) Pub. No.: US 2010/0004214 A1
(43) Pub. Date: Jan. 7, 2010

(54) SEQUENTIAL SPRM/ PROGESTIN TREATMENT

(76) Inventor: Kristof Chwalisz, Mundelein, IL (US)

Correspondence Address: Robert DeBerardine;Abbott Laboratories, D-377/AP6A-1, 100 Abbott Park Road, Abbott Park, IL, 60064-6008, US

(21) Appl. No.: 12/558,728

(22) Filed: Sep. 14, 2009

Related U.S. Application Data
(63) Continuation of application No. 11/089,275, filed on Mar. 24, 2005.
(60) Provisional application No. 60/555,917, filed on Mar. 24, 2004.

(51) Int. Cl. A61K 31/56

(52) U.S. Cl. 514/170; 514/171

(57) ABSTRACT

The methods provided herein comprise administering a selective progesterone receptor modulator (SPRM) during a first dosing period and at least one progestogen during a second dosing period. The dosing periods can run concomitantly or sequentially with or without a period where neither the SPRM nor the progestogen is administered.

### FIELD OF THE INVENTION

This invention relates to selective progesterone receptor modulators (SPRMs), and in particular relates to the use of SPRMs in combination with an agent for predictably inducing menstrual bleeding.

### BACKGROUND OF THE INVENTION

Female reproductive functions are characterized by variable cycles. The beginning and the end of these cycles are determined by the shedding and expulsion (sloughing) of superficial layers of the endometrium. Ovarian hormones, mostly progesterone, control menstruation, but local pro-inflammatory mediators such as prostaglandines and nitric oxide play an important role in this event as well. Menstruation is initiated by the constriction of spiral arteriols in the endometrium. The expulsion of the superficial layers of the endometrium from the uterus is brought about by uterine contractions that increase in intensity and duration during menstruation. This process is accompanied by bleeding, due to vascular breakdown, dilatation of vessels and increased fibrinolitic activity in the menstrual endometrium. In the absence of pregnancy, the normal duration of the menstrual cycle is 28 days. During pregnancy, the amenorrhea period lasts approximately 40 weeks, despite the presence of very high estrogen and progesterone concentrations. If lactation follows parturition, this may also lead to an even longer state of amenorrhea.

For a variety of therapeutic reasons, it is desirable to alter or stop the normal menstruation cycle and thereby induce a condition known as amenorrhea. Therapeautic regimens that combine the administration of gonadotropin releasing hormone and progesterone are known for various gynecological disorders that are improved by the induction of amenorrhea. These regimens are, however, associated with clinically relevant side effects. Gonadotropin releasing hormone analogs lead to estrogen deprivation, which is associated with hot flashes and bone loss. For reasons yet unknown, it has not been possible to induce complete amenorrhea with hormonal regimens including administration of a continuous oral contraceptive and a progestogen. Characteristically, these regimens lead to breakthrough bleedings or spotting in a substantial percentage of patients. The unscheduled uterine bleeding associated with chronic hormonal regimens has a negative impact on a patient acceptability.

SPRMs suppress endometrial proliferation and bleeding and induce long term amenorrhea. The effects of SPRMs are exerted at the endometrial level, i.e., without affecting the ovarian estrogen secretion (Chwalisz K, Elger W, McCrary K, Beckman P, Larsen L. Reversible suppression of menstruation in normal women irrespective of the effect on ovulation with the novel selective progesterone receptor modulator (SPRM) J867 J Soc Gynecol Invest 2002; 9 (Suppl) (1): Abstract 49). However, the perception of therapeutically induced long term amenorrhea varies in different cultures. Moreover, this perception also is dependent on socio-economical status and age. For example, some women clearly prefer reducing the number of uterine bleeding episodes per year instead of completely inducing amenorrhea. Stopping SPRM therapy allows a patient to return to a menstrual cycle. Unfortunately, however, terminating amenorrhea induced by SPRM treatment results in withdrawal bleeding that occurs at various and unpredictable times.

In a study about the effect of SPRM inducing amenorrhea in premenopausal women (Chwalisz K, Lamar Parker R, Williamson S, Larsen L, McCrary K, Elger W. treatment of uterine leiomyomas with the novel selective progesterone receptor modulator (SPRM) J867. J Soc Gynecol Invest 10,2: abstract 636) it was shown that the withdrawal bleeding, which started the spontaneous menstrual cycle, occurred up to 90 days after stopping the SPRM treatment.

In the majority of subjects, the resumption of menstruation occurred within 30 days of the cessation of treatment. However, a substantial number of women experienced longer intervals. Hence, while a return to menstruation is possible for women taking SPRM therapy for purposes of inducing amenorrhea, the predictability of a return to a menstrual cycle is variable.

There is therefore an need for a therapy that provides a condition of reliable amenorrhea and a predictable return to menstruation. It would further be desirable if such a therapy provided the above objectives without the side effects associated with previous therapies designed to induce amenorrhea.

### SUMMARY OF THE INVENTION

The present invention provides methods and kits that can be used to reliably induce amenorrhea and a subsequent return to menstruation. These methods and kits allow patients to return to menstrual bleeding after a period of amenorrhea. The methods generally can be used to regulate a patients menstrual cycle and for treat gynaecological disorders. Generally, the methods comprise administering a selective progesterone receptor modulator (SPRM) during a first dosing period and at least one progestogen during a second dosing period. The dosing periods can run concomitantly or sequentially with or without a period where neither the SPRM nor the progestogen is administered. The second dosing period may further comprise the administration of an estrogen.

### DETAILED DESCRIPTION OF THE INVENTION

SPRMs (“variously referred to as “mesoprogestins”) are a class of progesterone receptor ligands that possess partial agonistic and antagonistic activity in animals and humans. SPRMs show a high degree of endometrial selectivity and control of endometrial function without compromising ovarian estrogen production and thus do not induce estrogen deficiency. The antagonistic activity of SPRMs is incomplete inasmuch as SPRMs will not, in effect, completely block progesterone action as observed with progesterone antagonists such as mifepristone (RU486). Hence, SPRMs have incomplete progesterone receptor antagonist activity due to the fact that they also display high levels of intrinsic agonist activity. From a more quantitative standpoint, SPRMs score lower than progesterone in the McPhail bioassay in rabbits, but considerably higher than progesterone antagonists, such as mifepristone or onapristone, that do not show any agonist activity in this test. McPhail tests are widely used to semiquantitatively assess agonistic and antagonistic effects of compounds at the progesterone receptor using a rabbit model. The McPhail test uses a scale of 0-4 with progesterone having the highest score of 4. RU486, on the other hand, has a score of less than 0.5 and is therefore considered a pure antagonist in this model. The McPhail test is described in Selye H., Textbook of Endocrinology, 1947, pp 345-346. McPhail testing and in vivo characterization readily can be used to identify SPRMs. Using the McPhail test as a guide, SPRMs generally can be categorized as compounds having a McPhail score of between 0.5 and 3.5, more preferably between 0.5 and 3, and most preferably between 0.5 and 2. Compounds having such activities, as well as methods for synthesizing such compounds, have been described in U.S. Pat. Nos. 5,843,931; 5,519,027; 5,426,102; 5,244,886; 5,273,971; 5,446,063; 5,576,310 and 5,693,628; (all of which are herein incorporated by reference) as well as in PCT Patent Applications having publication numbers WO 01/26603; WO 01/34126; and WO 01/15679. Compounds that have previously been designated J867, J900, J956, J912, J914, and J1042 are all suitable for use in accordance with the methods provided herein. Such compounds include [4-[17β-Methoxy-17α-(methoxymethyl)-3-oxoestra-4,9-dien-11β-yl]benzaldehyd-(1E)-oxim]; [4-17β-Hydroxy-17α-(methoxymethyl)-3-oxoestra-4,9-dien-11β-yl]benzaldehyd-(1E)-oxim]; [4-17β-Methoxy-17α-(methoxymethyl)-3-oxoestra-4,9-dien-11β-yl]benzaldehyd-(1E)-[O-(ethoxy)carbonyl]oxim; [4-17β-Methoxy-17α-(methoxymethyl)-3-oxoestra-4,9-dien-11β-yl]benzaldehyd-(1E)-(O-acetyl)oxim]; and [4-[17β-Methoxy-17α-(methoxymethyl)-3-oxoestra-4,9-dien-11β-yl]benzaldehyd-(1E)-[O-(ethylamino)carbonyl)oxim].

As mentioned above, SPRMs can induce amenorrhea and have been indicated for a variety of gynaecological disorders including, for example, uterine fibroids, endometriosis, hormone replacement therapy, menorrhagia, metrorrhagia, dysmenorrhea, adenomyosis, and peritoneal adhesions. As previously mentioned, the present invention permits a predictable induction of menstrual bleeding after a period of amenorrhea resulting from SPRM therapy. Hence, the methods and kits provide a means for generating long term menstrual cyclicity where predictable, and when desired, prolonged periods of amenorrhea are followed by a predictable cycle of menstrual bleeding. Accordingly, the methods provided herein can be employed in connection with any of the above indications or subsequently discovered indications where amenorrhea is induced and a predictable return to menstruation is desired. The methods and kits are particularly suited for therapeutic indications such as endometriosis, fibroids, and uterine bleeding.

According to the methods provided herein SPRM therapy is given during a first dosing period. A “dosing period” as used herein is a period of time, such as for example, days, weeks, or months where a patient takes a given medication. The medication may be given one or more times a day. Typically, the first dosing period will range from about 1 month to about 12 months, more typically from about 3 to 12 months, and even more typically, for about 3, about 6, or about 12 months. Advantageously, the exact time period for the first dosing period is actually a matter of choice for a medical professional or even a patient based upon the length of time they subjectively choose to extend the period of amenorrhea. Hence, a medical professional or patient has the opportunity to regulate and select the onset of menstrual bleeding by shortening or prolonging the phase where SPRM therapy is administered.

During the first dosing period, a therapeutically effective amount of SPRM is administered to a patient in need of the therapy provided by the present methods and kits. The phrase “therapeutically effective amount” as used herein means a sufficient amount of, for example, a composition, compound, or formulation necessary to treat the desired disorder, at a reasonable benefit/risk ratio applicable to any medical treatment. As with other pharmaceuticals, it will be understood that the total daily usage of SPRMs or other drugs mentioned herein will be decided by a patient's attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved. These parameters can then be employed to appropriately dose a particular patient such that a patient receives the desired effect. Preferably, the SPRM is administered in a single or divided dose of 0.125 mg and 100 mg per day, more preferably between 1 mg and 50 mg per day.

During the second dosing period, a therapeutically effective amount of at least one progestogen is administered to a patient in need of the therapy provided by the present methods and kits. The second dosing period is usually measured in days and is typically between 1 day and 30 days, more preferably between 5 days and 20 days, and most preferably between 7 days and 14 days. Progestogens include any compound capable of inducing a withdrawal menstruation including progesterone and synthetic progestins. Such compounds would include progestogens such as, for example, medroxyprogesterone, cyproterone acetate, drospirenone, dydrogesterone, dienogest, noresthisterone, levonorgestrel, gestodene, promegestone, and trimegestone. In addition to the progestogen, an estrogen such as, for example, ethinylestradiol, conjugated equine estrogens, or biogene estrogens including estradiol, estriol, estrone or their esters may optionally be administered during the second dosing period.

Generally, the daily dose of the progestogen will be in the range of between 0.01 mg to 100 mg/day. More preferred doses of particular progestogens include but are not limited to, 5-10 mg/day medroxyprogesteron acetate, 1-3 mg/day cyproterone acetate, 1-5 mg/day drospirenone, 10-20 mg/day dydrogesterone, 1-6 mg/day dienogest, 0.5-2 mg/day noresthisterone acetate or norethisterone, 0.05 mg/day levonorgestrel, 0.05-0.2 mg/day gestodene are suitable doses.

The daily oral dose of an estrogen, such as estradiol, is preferably between 0.3 mg and 3.0 mg per day, and more preferably between 0.5 mg and 2.0 mg/day; or an equivalent dose of estradiol, provided in an alternative administration route; or an equivalent dose of other biogen estrogens. Conjugated equine estrogens are preferably dosed between 0.3 mg and 5 mg per day. Other specific estrogens and their doses include ethinylestradiol at dosages from 10 to 100 μg/day, preferably 15-30 μg/day.

As explained more fully below, estrogens can be administered transdermally or vaginally. The non-oral administration of estrogens preferably releases approximately 10 μg/day to 50 μg/day of 17β-estradiol or a bioequivalent amount of another estrogen, on a daily basis.

The first and the second dosing periods may run sequentially or concomitantly. For example, the first dosing period may end and the second dosing period can begin the next day after the end of the first dosing period. Alternatively, there may be one or more days between the end of the first dosing period and the beginning of the second dosing period where no medication or a placebo is administered to a patient in need of the therapy provided by the methods and kits provided herein. As mentioned above, the dosing periods may run concomitantly or overlap for one or more days. For example, the second dosing period may begin prior to the end of the first dosing period. Hence, during such an overlap between the first and second dosing periods, a SPRM and a progestogen would be administered to the patient.

SPRMs, as well as the progestogen, can be administered in a variety of forms. Compounds of this invention may be administered orally, ophthalmically, osmotically, parenterally (subcutaneously, intramuscularly, intrasternally, intravenously), rectally, topically, transdermally, or vaginally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous suspensions, which suspensions comprise crystalline, amorphous, or otherwise insoluble forms of the compounds. Rectally and vaginally administered compounds may be administered as creams, gels, lotions, ointments, and pastes.

Depending upon the form of administration, SPRMs and the progestogen, may be formulated or administered with or without a pharmaceutically acceptable excipient. Such excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof.

For example, excipients for orally administered compounds in solid dosage forms include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodium phosphate salts, soybean oil, sucrose, tetrahydrofurfuryl alcohol, and mixtures thereof. Excipients for ophthalmically and orally administered compounds in liquid dosage forms include benzyl alcohol, benzyl benzoate, 1,3-butylene glycol, castor oil, corn oil, cottonseed oil, ethanol, ethyl acetate, ethyl carbonate, fatty acid esters of sorbitan, germ oil, groundnut oil, glycerol, isopropanol, olive oil, polyethylene glycols, propylene glycol, sesame oil, tetrahydrofurfuryl alcohol, water, and mixtures thereof. Excipients for osmotically administered compounds include chlorofluorohydrocarbons, ethanol, isopropanol, water, and mixtures thereof. Excipients for parenterally administered compounds include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for rectally and vaginally administered compounds include cocoa butter, polyethylene glycol, wax, and mixtures thereof.

The phrase “pharmaceutically acceptable” as used herein includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

SPRMs and the progestogen may separately be provided or packaged as kits. Advantageously, each compound of the kit may be packaged in per use groupings such that, for example, a daily prescription of each component can identified in order to enhance patient compliance. Sets of the compounds may be identified in a variety of ways. For example, a set of compounds may be identified on the package containing the compounds. Alternatively, external instructions may be provided with a set or sets of the compounds that, for example, identify a grouping and instruct a patient appropriate times to take the components of the kit. Convenience packs, such as those described above, are well known and take a variety of forms such as, for example, those described in U.S. Pat. Nos. 3,921,804; 4,964,539; 5,316,400; and 5,775,536. So-called “blister packs” are a common type of convenience pack and generally comprise a sheet of material that can be formed with blisters to contain a solid dosage form and a backing sheet sealed to the blistered material to maintain the dosage form in the individual blisters.

Variations and changes which are obvious to one skilled in the art are intended to be within the scope of the invention.

1. A method for regulating a menstrual cycle comprising administering a selective progesterone receptor modulator during a first dosing period and at least one progestogen during a second dosing period.

2. The method of claim 1 wherein the first dosing period is between about 1 month and about 12 months.

3. The method of claim 2 wherein the second dosing period is between 1 day and 31 days.

4. The method of claim 3 wherein the second dosing period begins the first day after the first dosing period ends.

5. The method of claim 1 wherein the first dosing period and second dosing period overlap for at least one day.

6. The method of claim 1 wherein the SPRM is administered in an amount between 0.125 mg and 100 mg per day during the first dosing period.

7. The method of claim 6 wherein the progestogen is administered in an amount between 0.01 mg and 100 mg per day during the second dosing period.

8. The method of claim 1 wherein the SPRM is selected form the group consisting of 11β-[4-(hydroxyiminomethyl)phenyl]-17β-methoxy-17α-(methoxymethyl)estra-4,9-dien-3-one (asoprisnil), 11β-[4-(hydroxyiminomethyl)phenyl]-17β-hydroxy-17α-(methoxymethyl)estra-4,9-dien-3-one (J912), and 11β-[4-[(ethylaminocarbonyl)oximinomethyl]phenyl]-17β-methoxy-17α-(methoxymethyl)estra-4,9-dien-3-one (J956) as well as pharmaceutically acceptable salts thereof.

9. The method of claim 1 wherein the progestogen is selected from the group consisting of medroxyprogesterone, cyproterone, drospirenone, dydrogesterone, dienogest, noresthisterone, levonorgestrel, gestodene, promegestone, trimegestone, and pharmaceutically acceptable salts thereof.

10. The method of claim 9 wherein the method further comprises administering an estrogen during the second dosing period.

11. A method of treating a gynaecological disorder comprising administering to a patient a SPRM for a first dosing period, wherein the improvement comprises administering at least one progestogen during a second dosing period.

12. The method of claim 11 wherein the first dosing period is between about 1 month and 12 months and the second dosing period is between 1 day and 31 days and the second dosing period begins the day following the first dosing period.

13. The method of claim 12 wherein the SPRM is selected form the group consisting of 11β-[4-(hydroxyiminomethyl)phenyl]-17β-methoxy-17α-(methoxymethyl)estra-4,9-dien-3-one (asoprisnil), 11β-[4-(hydroxyiminomethyl)phenyl]-17β-hydroxy-17α-(methoxymethyl)estra-4,9-dien-3-one (J912), and 11β-[4-[(ethylaminocarbonyl)oximinomethyl]phenyl]-17β-methoxy-17α-(methoxymethyl)estra-4,9-dien-3-one (J956) as well as pharmaceutically acceptable salts thereof.

14. A kit comprising a SPRM and at least one progestogen.

15. The kit of claim 14 wherein the SPRM is selected form the group consisting of 11β-[4-(hydroxyiminomethyl)phenyl]-17β-methoxy-17α-(methoxymethyl)estra-4,9-dien-3-one (asoprisnil), 11β-[4-(hydroxyiminomethyl)phenyl]-17β-hydroxy-17α-(methoxymethyl)estra-4,9-dien-3-one (J912), and 11β-[4-[(ethylaminocarbonyl)oximinomethyl]phenyl]-17β-methoxy-17α-(methoxymethyl)estra-4,9-dien-3-one (J956) as well as pharmaceutically acceptable salts thereof; and the progestogens are selected from the group consisting of progesterone and any other synthetic progestin as well as their pharmaceutically acceptable salts and combinations of the foregoing.
